(19) United States
(12) Patent Application Publication
(10) Pub. No.: US 2005/0054688 A1
(43) Pub. Date: Mar. 10, 2005

(54) Selective serotonin reuptake inhibitors in the treatment of disease

(75) Inventors: Kathryn Elizabeth May, County of Kent (GB); Paul Quinn, County of Kent (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/911,806

(22) Filed: Aug. 5, 2004

Related U.S. Application Data
(60) Provisional application No. 60/528,136, filed on Dec. 9, 2003.

(30) Foreign Application Priority Data
Aug. 8, 2003 (GB) 0318706.9

(51) Int. Cl. A61K 31/44; A61K 31/18; A61K 31/137

(52) U.S. Cl. 514/345; 514/602; 514/649

(57) ABSTRACT

The present invention provides the use of selective serotonin reuptake inhibitors (SSRIs) to treat premature female orgasm.

This application claims priority from United Kingdom Application Number 0318706.9, filed on Aug. 8, 2003 and the benefit of U.S. Provisional Application No. 60/528,136, filed on Dec. 9, 2003.

This invention relates to the use of compounds which exhibit activity as selective serotonin re-uptake inhibitors (SSRIs) to treat premature female orgasm, and to pharmaceutical formulations containing such compounds.

Whilst anorgasmia and difficulties with orgasm are well-represented in the female sexual dysfunction literature, rapid orgasm—a female problem, sharing components with premature ejaculation (PE) in men—is notable by its absence. DSMIVR (2000) includes no mention of it, although it has an otherwise comprehensive section on orgasmic disorders.

It has been suggested that the distribution curve for female orgasm is likely to be very similar to that used to describe male experience (Cole and Dryden, 1988). However, both clinically and in the research and its literature, the ‘retarded’ as opposed to the ‘premature’ end of this is very much the focus of interest. This weighting is also strongly reflected in cases seen, with cases of rapid orgasm in women presenting rarely and representing a far smaller percentage of the female caseload than the 30% or more of male caseload taken by PE. This is partly because male partners often choose to take rapid orgasm as positive feedback on their skill as a lover and partly because, generally speaking, it presents fewer problems in terms of continuing with intercourse than PE. This is particularly so where the female partner is multi-orgasmic and therefore potentially well-motivated to continue.

Unfortunately, a proportion of women with rapid orgasm find continued post-orgasmic stimulation uncomfortable, if not painful. This group has no wish to continue with intercourse, having reached orgasm, and indeed may feel unable to do so. This tends to give rise to interpersonal distress, which is difficult to resolve and these tend to be the couples referred to Sex Therapy Clinics and gynaecology. The problem of ‘disengagement’ of the female partner is also reported, with males feeling incidental or rejected, in the same way as female partners of men with PE.

Cognitive/behavioural treatments are more difficult to describe and implement than in PE, the arousal element of the Human Sexual Response curve being so attenuated that women find it immensely difficult to assert any kind of control. Whilst men with PE are educated to recognise the ‘point of ejaculatory inevitability’, a corresponding ‘point of orgasmic inevitability’ is not identified and intervention to disrupt it would anyway presumably prove to be problematic. The adoption of other treatment strategies used in PE (SS cream, local anaesthetics, SSRIs) is not reported in the small available literature on rapid orgasm in women.

Premature female orgasm may be defined as:
a) Persistent or recurrent orgasm with minimal sexual stimulation, occurring before, upon or shortly after penetration and generally before the person/couple wishes it.
b) Interpersonal difficulties arise from female lack of motivation for/discomfort with continued sexual stimulation.
c) The disturbance gives rise to negative psychosocial consequences where arousal occurs in inappropriate situations
d) The disturbance itself causes marked distress or interpersonal difficulty

According to a first aspect, the invention provides for a selective serotonin reuptake inhibitor in the preparation of a medicament for the treatment or prevention of premature female orgasm.

By SSRI it is meant a compound with a serotonin re-uptake inhibition (SRI) IC₅₀ value of less than or equal to 100 nM. Preferred compounds have a serotonin re-uptake inhibition (SRI) IC₅₀ value of less than or equal to 50 nM. Particularly preferred compounds have a serotonin re-uptake inhibition (SRI) IC₅₀ value of less than or equal to 10 nM.

In a preferred embodiment the compounds are more than 10-fold as potent in the inhibition of serotonin re-uptake than in the inhibition of dopamine re-uptake, preferred compounds are more than 100-fold as potent, particularly preferred compounds are more than a 1000-fold as potent.

In a preferred embodiment the compounds are more than 10-fold as potent in the inhibition of serotonin re-uptake than in the inhibition of noradrenaline re-uptake, preferred compounds are more than 100-fold as potent, particularly preferred compounds are more than a 1000-fold as potent.

Particularly suitable SSRIs for use in treating premature female orgasm may be characterised by having a rapid onset and a short duration of action.

Suitable SSRIs for use in the present invention include: sertraline, fluoxetine, fluvoxamine, paroxetine, citalopram, dapoxetine, 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), N-methyl-N-({3-[3-methyl-4-(methylsulfanyl)phenoxy]-4-pyridinyl}methyl)amine (Example 38, PCT Application no PCT/IB02/01032), methyl-[3-(4-methylsulfanyl-phenoxy)-pyridin-4-ylmethyl]amine (Example 35 WO02/083643), Escitalopram, Litoxetine, 403U76, VN-2222, Roxindle, Milnacipran, RS-1439, R-fluoxetine, WF-32, DOV-216303, LY-393558, LY-433221, Cericlamine, UP-23761, Indalpine, YM-992/Lubazodone, A-80426, Nefazodone, DuP-631, Bicifadine, Duloxetine, LY-214821, LY-393558, LU-10134-C, Sibutramine, SPD-473, Tramadol, CI-275838, McN-5652, L792239, Vilazodone, NS-2359, NS-2381, ORG-6582, Femoxetine, RS-1259, R-DDMS, S-sibutramine, S-fluoxetine, Dexenfuramine, S-34324, Du-125530, SLV-210, SLV-313, SLV-314 and Venlafaxine.

Preferred SSRIs are sertraline, fluoxetine, fluvoxamine, paroxetine, citalopram, dapoxetine, 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), methyl-[3-(4-methylsulfanyl-phenoxy)-pyridin-4-ylmethyl]amine (Example 35 WO02/083643), N-methyl-N-({3-[3-methyl-4-(methylsulfanyl)phenoxy]-4-pyridinyl}methyl)amine (Example 38, PCT Application no PCT/IB02/01032).

More preferred SSRIs are 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), methyl-[3-(4-methylsulfanyl-phenoxy)-pyridin-4-ylmethyl]amine (Example 35 WO02/083643), N-methyl-N-({3-[3-methyl-4-(methylsulfanyl)phenoxy]-4-pyridinyl}methyl)amine (Example 38, PCT Application no PCT/IB02/01032), paroxetine and dapoxetine.

Yet more preferred are 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), methyl-[3-(4-methylsulfanyl-phenoxy)-pyridin-4-ylmethyl]amine (Example 35 WO02/083643), N-methyl-N-({3-[3-methyl-4-(methylsulfanyl)phenoxy]-4-pyridinyl}methyl)amine (Example 38, PCT Application no PCT/IB02/01032).

The text of the aforementioned patents and all other references cited in this specification are hereby incorporated by reference in their entirety.

It should be understood that compounds suitable for use in the present invention, as exemplified above or as identified by the assays described herein, may encompass pharmaceutically acceptable salts, solvates and prodrugs thereof.

Compounds suitable for use as an SSRI may be identified by the use of the following assay:

Biological Activity

Compounds may be tested for their ability to inhibit the uptake of serotonin by human serotonin transporters, dopamine by human dopamine transporters or nopradrenaline by human noradrenaline transporters as follows.

(i) Cell Culture

Human embryonic kidney cells (HEK-293) stably transfected with either the human serotonin transporter (hSERT), noradrenaline transporter (hNET) or dopamine transporter (hDAT) were cultured under standard cell culture techniques (cells were grown at 37° C. and 5% CO₂ in DMEM-culture media (supplemented with 10% dialysed foetal calf serum (FCS), 2 mM l-glutamine and 250 μg/ml geneticin)). Cells were harvested for the assay to yield a cell suspension of 750,000 cells/ml.

(i) Determination of Inhibitor Potency

All test compounds were dissolved in 100% dimethylsulfoxide (DMSO) and diluted down in assay buffer to give appropriate test concentrations. Assays were carried out in 96-well filter bottom plates. Cells (7500 cells/assay well) were pre-incubated in standard assay buffer containing either test compound, standard inhibitor or compound vehicle (1% DMSO) for 5 minutes. Reactions were started by addition of either ³H-Serotonin, ³H-Noradrenaline or ³H-Dopamine substrates. All reactions were carried out at room temperature in a shaking incubator. Incubation times were 5 minutes for the hSERT and hDAT assays and 15 minutes for the hNET assay. Reactions were terminated by removal of the reaction mixture using a vacuum manifold followed by rapid washing with ice cold assay buffer. The quantity of ³H-substrate incorporated into the cells was then quantified.

Assay plates were dried in a microwave oven, scintillation fluid added, and radioactivity measured. Potency of test compounds was quantified as IC₅₀ values (concentration of test compound required to inhibit the specific uptake of radiolabelled substrate into the cells by 50%).

(iii) Standard Assay Buffer Composition:

Trizma hydrochloride (26 mM)
NaCl (124 mM)
KCl (4.5 mM)
KH₂PO₄ (1.2 mM)
MgCl₂.6H₂O (1.3 mM)
Ascorbic acid (1.136 mM)
Glucose (5.55 mM)
pH 7.40
CaCl₂ (2.8 mM)
Pargyline (100 μM)

Note: The pH of the buffer was adjusted to 7.40 with 1M NaOH before addition of CaCl₂ and pargyline.

(iv) Summary of Assay Parameters

| | hSERT | hDAT | hNET |
|---|---|---|---|
| | Assay | Assay | Assay |
| Cell concentration per | 75,000 | 75,000 | 75,000 |
| assay well. | | | |
| Substrate Concentration. | ³H-5HT | ³H-Dopamine | ³H-Noradrenaline |
| | (50 nM) | (200 nM) | (200 nM) |
| Incubation time (minutes) | 5 | 5 | 15 |

The compounds of the invention have the advantage that they are selective inhibitors of the re-uptake of serotonin (SRIs) (and so are likely to have reduced side effects), they have a rapid onset of action (making them suitable for administration shortly before an effect is required), they have desirable potency and associated properties. Compounds that selectively inhibit the re-uptake of serotonin, but not noradrenaline or dopamine, are preferred.

It will be appreciated by those skilled in the art that certain protected derivatives of compounds of the invention, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention.

All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference).

It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds of the invention.

Thus, according to further aspects, the invention provides:
i) the use of an SSRI in the manufacture of a medicament for the treatment or prevention of premature female orgasm.
ii) a method of treatment or prevention of premature female orgasm, which comprises administering a therapeutically effective amount of an SSRI to a patient in need of such treatment or prevention;

It is to be appreciated that all references herein to treatment include curative, palliative and prophylactic treatment.

The compounds of the invention may be administered alone or as part of a combination therapy. If a combination of active agents are administered, then they may be administered simultaneously, separately or sequentially. In particular, the compounds of the invention may be combined with the following preferably for the treatment of premature female orgasm:
i) Alpha-blockers (e.g. phentolamine, doxazasim, tamsulosin, terazasin, prazasin and Example 19 of WO9830560.
ii) Apomorphine—teachings on the use of apomorphine as a pharmaceutical may be found in U.S. Pat. No. 5,945,117.
iii) Dopamine D2 agonists (e.g. Premiprixal, Pharmacia Upjohn compound number PNU95666).
iv) Melanocortin receptor agonists (e.g. Melanotan II).
v) PGE1 receptor agonists (e.g. alprostadil).
i) Mono amine transport inhibitors, particularly Noradrenaline Re-uptake Inhibitors (NRIs) (e.g. Reboxetine), other Serotonin Re-uptake Inhibitors (SRIs) (e.g. paroxetine) or Dopamine Re-uptake Inhibitors (DRIs).
vii) 5-HT3 antagonists (e.g. ondansetron and granisetron).
viii) PDE inhibitors such as PDE2 (e.g. erythro-9-(2-hydroxyl-3-nonyl)-adenine) and Example 100 of EP 0771799-incorporated herein by reference) and in particular a PDE5 inhibitor (e.g. sildenafil, 1-{[3-(3,4-dihydro-5-methyl-4-oxo-7-propylimidazo[5,1-f]-as-trazin-2-yl)-4-ethoxyphenyl]sulfonyl}-4-ethylpiperazine i.e. vardenafil/Bayer BA 38-9456 or IC351 (see structure below, Icos Lilly)). 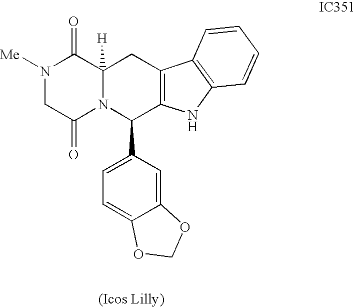
ix) Potassium channel openers.
x) P2X purinergic receptor antagonists.
xi) Endothelin receptor antagonists
xii) One or more of an androgen such as androsterone, dehydro-androsterone, testosterone, androstanedione and a synthetic androgen;
xiii) Oxytocin antagonists

For human use the compounds of the invention can be administered alone but in human therapy will generally be administered in admixture with a suitable pharmaceutical excipient, diluent or carrier selected with regard to the intended route of administration and standard pharmaceutical practice.

For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

### EXAMPLE

### Tablet Formulation

In general a tablet formulation could typically contain between about 0.01 mg and 500 mg of a compound of the invention whilst tablet fill weights may range from 50 mg to 1000 mg. An example formulation for a 10 mg tablet is illustrated:

| Ingredient | % w/w |
|---|---|
| Compound of the invention | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

For oral or parenteral administration to human patients the daily dosage levels of compounds of the invention will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred. Preferably, administration takes place shortly before an effect is required.

Thus according to a further aspect, the invention provides a pharmaceutical formulation containing a compound of the invention and a pharmaceutically acceptable adjuvant, diluent or carrier.

1.) A method of treating or preventing premature female orgasm comprising administering a therapeutically effective amount of a selective serotonin reuptake inhibitor to a patient in need of such treatment or prevention.

2) The method of claim 1 wherein the compound has a serotonin re-uptake inhibition (SRI) IC₅₀ value of less than or equal to 50 Nm.

3) The method of claim 1 or 2 wherein the compounds are more than 100-fold as potent in the inhibition of serotonin re-uptake than in the inhibition of dopamine re-uptake.

4) The method of claims 1 and 2 wherein the compounds are more than 10-fold as potent in the inhibition of serotonin re-uptake than in the inhibition of noradrenaline re-uptake.

5) The method of claim 1 wherein the selective serotonin reuptake inhibitor is selected from:
sertraline, fluoxetine, fluvoxamine, paroxetine, citalopram, dapoxetine, 3-[(dimethylamino)methyl]-4-[4-(methylsulfanyl)phenoxy]benzenesulfonamide, 3-[(dimethylamino)methyl]-4-[3-methyl-4-(methylsulfanyl)phenoxy]benzenesulfonamide, N-methyl-N-({3-[3-methyl-4-(methylsulfanyl)phenoxy]-4-pyridinyl}methyl)amine, Escitalopram, Litoxetine, Roxindle, Milnacipran, R-fluoxetine, Cericlamine, Indalpine, methyl-[3-(4-methylsulfanyl-phenoxy)-pyridin-4-ylmethyl]amine, Lubazodone, Nefazodone, Bicifadine, Duloxetine, Sibutramine, Tramadol, Vilazodone, Femoxetine, S-sibutramine, S-fluoxetine, Dexenfuramine, and Venlafaxine.

6) The method of claim 5 wherein the selective serotonin reuptake inhibitor is selected from
3-[(dimethylamino)methyl]-4-[4-(methylsulfanyl)phenoxy]benzenesulfonamide,
3-[(dimethylamino)methyl]-4-[3-methyl-4-(methylsulfanyl)phenoxy] benzenesulfonamide,
N-methyl-N-({3-[3-methyl-4-(methylsulfanyl)phenoxy]-4-pyridinyl}methyl)amine, and methyl-[3-(4-methylsulfanyl-phenoxy)-pyridin-4-ylmethyl]amine.
